(19) United States
(12) Patent Application Publication
(10) Pub. No.: US 2004/0127468 A1
(43) Pub. Date: Jul. 1, 2004

(54) Methods and compositions for treating apoptosis associated disorders

(76) Inventor: William George Tatton, Fort MacLeod (CA)

Correspondence Address: LAHIVE & COCKFIELD, LLP., 28 STATE STREET, BOSTON, MA, 02109, US

(21) Appl. No.: 10/414,810

(22) Filed: Apr. 16, 2003

Related U.S. Application Data
(63) Continuation of application No. 10/224,302, filed on Aug. 20, 2002.
(60) Provisional application No. 60/313,840, filed on Aug. 20, 2001.
(60) Provisional application No. 60/316,327, filed on Aug. 30, 2001.

(51) Int. Cl. A61K 31/66

(52) U.S. Cl. 514/121

(57) ABSTRACT

Methods for modulating apoptosis by administering a phosphatidic acid compound are described.

### RELATED APPLICATIONS

This application claims priority to U.S. Provisional Application Serial No. 60/316,327, entitled “Methods and Compositions for Treating Apoptosis Associated Disorders” filed on Aug. 30, 2001, and U.S. Provisional Application Serial No. 60/313,840, entitled “Methods and Compositions for Treating Apoptosis Associated Disorders” filed on Aug. 20, 2001; the entire contents of both of which are hereby incorporated herein by reference.

### BACKGROUND OF THE INVENTION

Over the last five years, apoptosis has been increasingly implicated in human disease. Apoptosis culminates in a number of controlled degradative events that produce membrane wrapped cell fragments, which are phagocytosed without inducing an inflammatory reaction. The degradative events are similar for many forms of apoptosis and include the reorganization and stripping of nuclear proteins from DNA, digestion of nuclear DNA by activated endonucleases, condensation of nuclear DNA, digestion of the cellular cytoskeleton and the formation of membrane wrapped cellular fragments often called apoptotic bodies.

Apoptosis can be characterized as involving three phases or stages: an initiation phase, a decisional phase and a degradative phase. Each phase involves series of signaling events that primarily involve protein interactions but also involve lipid moieties, changes in membrane permeability, ion fluxes, etc. Cysteine rich proteases called caspases play key roles in a number of initiation, decisional or degradative pathways. Also, changes in mitochondrial membrane permeability with the consequent release of factors that activate specific degradative pathways constitute a critical decisional event in some forms of apoptosis. In other forms of apoptosis, mitochondria are not involved.

Accordingly, apoptosis is not a single process. Rather, it can be visualized as involving a number of different, sometimes interconnected, signaling pathways leading to cell degradation. The pathways involved in a particular form of apoptosis depend on factors many factors such as the insult or insults that initiate the process (e.g., local ischemia, trophic insufficiency, etc.). Other factors include the activation or overactivation of specific receptors, such as the activation of “death” receptors by tumor necrosis factor alpha (TNFα) or FAS ligand and the overactivation iontotropic glutamate receptors (iGluR). Another determining factor is the type of cell which is involved, since different signaling pathways are shown for so called type I and type II cells after TNFα receptor activation.

At a simple level, two members of the BCL family of proteins can be seen to determine mitochondrial membrane permeability and therefore a decision to activate signaling for apoptotic degradation in those forms of apoptosis that involve mitochondria (see Jacotot E. et al. (1999) Ann N Y Acad Sci 887:18-30, for a review). Those proteins are generally BAX and BCL-2 (or its cousin BCL-XL). BAX can facilitate the opening of a membrane megapore that spans mitochondrial membranes and may also directly form pores in mitochondrial membranes. Either of those actions can increase mitochondrial membrane permeability and lead to release of mitochondrial degradative signaling factors. In contrast, BCL-2 retards opening of the megapore and may also prevent the formation of membrane pores, thereby maintaining mitochondrial membrane impermeability and decreasing the probability of apoptotic degradation.

In a non-disease state, a level of homeostasis exists which balances the apoptotic promoting and apoptotic inhibiting signals. When the apoptotic promoting signals are excessive, vital cells may die. For example, in Parkinson's disease, high levels of p53 and Fas ligand have been associated with neuron death. (Tatton, N. Experimental Neurology 166:29). Previously, work had suggested that phosphatidic acid may reduce apoptosis induced by the lipid moiety ceramide by inhibiting the ceramide-activated phosphatases that dephosphorylates Akt, a kinase whose phosphorylation can facilitate cell survival (Chalfant CE et al. (1999) J Biol Chem 274:20313-20317; Kishikawa K et al. (1999) J Biol Chem 274:21335-21341).

### SUMMARY OF THE INVENTION

In one embodiment, the invention pertains, at least in part, to methods of modulating apoptosis using phosphatidic acid compounds of the invention.

In another embodiment, the invention pertains to methods of treating an apoptosis associated disorder in a subject. The method includes administering a therapeutically effective amount of a phosphatidic acid compound of formula (I):

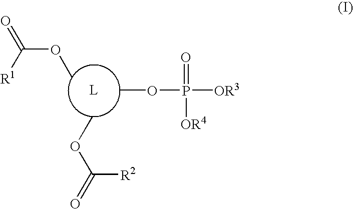

wherein

R¹ and R² are each independently selected chain moieties;

R³ and R⁴ are each independently hydrogen, absent, or a prodrug moiety;

L is a linking moiety, and pharmaceutically acceptable salts thereof.

In a further embodiment, the invention pertains also to a method of modulating apoptosis in a cell in vitro by administering an effective amount of a phosphatidic acid compound of formula (I).

In a further embodiment, the invention also pertains, at least in part, to a method for treating a neurodegenerative disorder in a subject. The method includes administering to a subject an effective amount of a phosphatidic acid compound of formula (I), (II), or (III), such that the neurodegenerative disorder is treated.

In another embodiment, the invention also includes a method for treating an eye disorder in a subject. The method includes administering to a subject an effective amount of a phosphatidic acid compound of formula (I), (II), or (III), such that the eye disorder is treated. In a further embodiment, the eye disorder is glaucoma.

The invention also pertains, at least in part, to methods for treating an apoptosis associated disorder in a subject, by administering to the subject an effective amount of a phosphatidic acid compound, e.g., a compound of formula (I), (II), or (III).

The invention also pertains, at least in part, to pharmaceutical compositions containing an effective amount of a phosphatidic acid compound (e.g., a compound of formula (I), (II), or (III)) and a pharmaceutically acceptable carrier.

In addition, the invention also pertains, at least in part to a packaged pharmaceutical composition. The packaged pharmaceutical compositions includes a phosphatidic acid compound of formula (I), or a pharmaceutically acceptable salt thereof, and instructions for the use of said compound for the treatment of a apoptosis associated state.

### BRIEF DESCRIPTION OF THE DRAWINGS

FIG. 1 shows a schematic drawing of some hypothesized apoptotic and anti-apoptotic signaling pathways.

FIGS. 2A, 2B, and 2C are graphs showing the survival of NGF differentiated PC12 cells (i.e. neuronally differentiated cells) treated with phosphatidic acid after exposure to C2-ceramide (FIG. 2A), rotenone (FIG. 2B) and peroxide (FIG. 2C).

FIGS. 3A, 3B, and 3C are line graphs which illustrate the increased survival of NGF differentiated PC12 cells treated with phosphatidic acid. FIG. 3A shows the results for cells exposed to rotenone. FIG. 3B shows the results of increased phosphatidic acid concentration on apoptosis relating to serum and NGF withdraw. FIG. 3C shows the affect of phosphatidic acid on peroxide induced apoptosis.

FIGS. 4A, 4B, 4C and 4D are line graphs which show that phosphatidic acid reduces the apoptosis-associated decrease of mitochondrial membrane potential (Δψ_{M}), a marker for increased mitochondrial membrane permeability, for both apoptosis initiated by ceramide (FIGS. 4A and 4B) and rotenone (FIGS. 4C and 4D). FIGS. 4A and 4C were determined by using the mitochondrial potentiometric dye TMRM. FIGS. 4B and 4D were obtained using the mitochondrial potentiometric dye CMTMR.

### DETAILED DESCRIPTION OF THE INVENTION

This invention is based, at least in part, on the discovery that phosphatidic acid is an anti-apoptotic agent for insults that are used as models for a number of human neurological diseases. Furthermore, it has also been discovered that phosphatidic acid decreases apoptosis induced by the sudden withdrawal of NGF and serum (trophic withdrawal), rotenone, and glutamate.

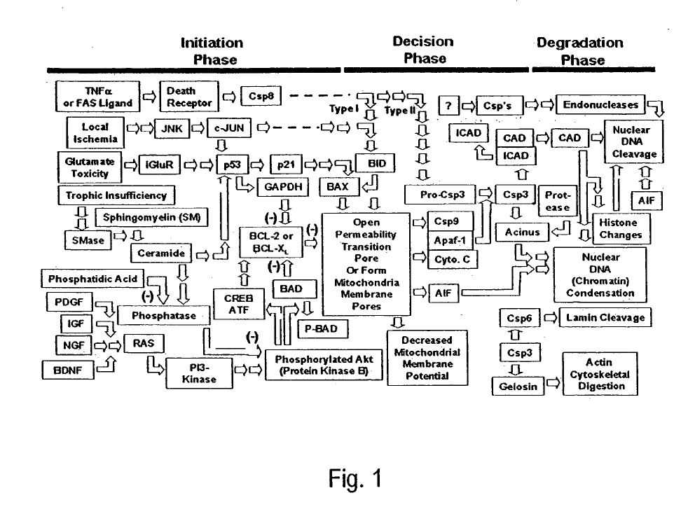

Apoptosis research has begun to reveal unique initiation and decisional signaling pathways that are responsible for apoptosis in a number of specific disease states. For example in Parkinson's disease (PD), signaling pathways involving p53, glyceraldehyde-3-phosphate dehydrogenase (GAPDH), BAX and Csp3 appear to contribute to the neuronal loss which underlies the disease (de la Monte SM et al. (1998) Lab Invest 78:401-411; Hartmann A et al. (2000) PNAS 97:2875-2880; Tatton NA, (2000) Exp Neurol 166:29-43). The rationale for determining the involvement of specific apoptosis signaling elements in different diseases relates to the possibility of developing pharmacological agents with the capacity to interrupt signaling by that element and thereby preventing the progression of a specific apoptosis pathway without effecting any untoward effects on normative cell function. FIG. 1 summarizes a number of apoptotic and anti-apoptotic signaling pathways.

Phosphatidic acid compounds of the invention were identified by studying the interactions between known anti-apoptotic propargylamines (AAPs) and GAPDH (Boulton A A (1999) Mech Ageing Dev 111:201-209). Propargylamines that are structurally similar to (−)-deprenyl, a monoamine oxidase B (MAO-B) inhibitor, have the capacity to reduce some forms of apoptosis independently of MAO-B inhibition (Tatton W G et al. (1991) J Neurosc Res 30:666-627; Ansari K S et al. (1993) J Neurosci 13:4042-4053; Tatton W G et al (1996) Neurol 47:S171-S183; Tatton W G et al. (1993) Monoamine Oxidase Inhibitors In Neurological Diseases (Lieberman A, ed). New York: Raven Press; Tatton W G et al. (1994) J Neurochem 63:1572-1575). GAPDH upregulation has been shown to be essential to some forms of apoptosis signaling (see (Tatton W G et al. (2000) J Neural Transm Suppl 60:77-100) for a review), and it has been shown that AAPs bind to GAPDH and convert it from a tetrameric form to a dimeric form (Kragten E et al. (1998) J Biol Chem 273:5821-5828; Carlile G W et al. (2000) Mol Pharmacol 57:2-12). The tetrameric form of GAPDH may be necessary for apoptosis signaling, because while the dimeric form induced by AAPs cannot signal for apoptosis but does retain the capacity to convert glucose to pyruvate (glycolysis) (Carlile G W et al. (2000) Mol Pharmacol 57:2-12).

Upregulated GAPDH has been found to induce apoptosis and reduce mitochondrial membrane potential (Δψ_{M}), a marker for increased mitochondrial membrane permeability. In addition, a p53-GAPDH pathway has been shown to decrease the new synthesis of BCL-2 and increases levels of BAX in mitochondria, which together may increase mitochondrial membrane permeability, and decrease Δψ_{M} and lead to the release of mitochondrial factors that signal for apoptotic degradation. It has been shown that AAPs prevent apoptotic decreases in Δψ_{M}, which is in keeping with their capacity to maintain BCL-2 levels (Wadia et al (1998) J. Neuroscience 18(3):932-947; Tatton et al., 1996, Neurology,4:s171-183; Tatton W G et al. (1994) J Neurochem 63:1572-1575).

Co-immunoprecipitation studies were carried out to determine what proteins bind to GAPDH. One protein which was identified was phospholipase D2 (PLD2), which is a constitutively active enzyme that converts phosphatidyl choline to phosphatidic acid (PA) and choline, as shown in Scheme 1. Therefore, PLD2 levels may be expected to control phosphatidic acid levels, and thus modulate in an intrinsic anti-apoptosis pathway.

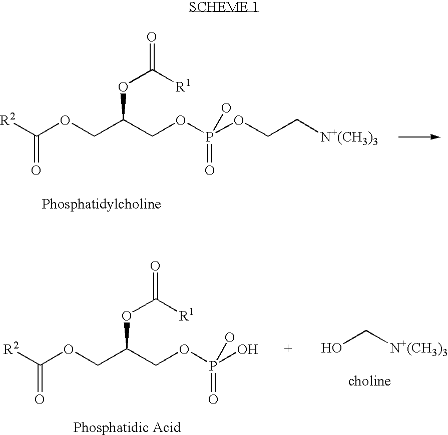

In an embodiment, the invention pertains, at least in part, to a method for treating an apoptosis associated disorder in a subject. The method includes administering a therapeutically effective amount of a phosphatidic acid compound of formula (I):

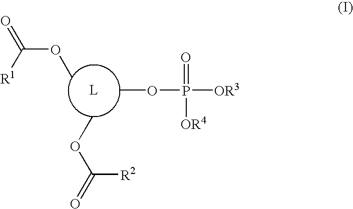

wherein

R¹ and R² are each independently selected chain moieties;

R³ and R⁴ are each independently hydrogen, absent, or a prodrug moiety;

L is a linking moiety, and pharmaceutically acceptable salts thereof.

The term “chain moiety” includes chains of atoms containing from one to thirty covalently linked atoms. The atoms may be substituted with hydrogen or one or more substituents which allow the phosphatidic acid compound to perform its intended function, e.g., modulate apoptosis. The chain moieties may include substituents which enhance their solubility or their cellular availability. Examples of chain moieties include, but are not limited to, chains of carbon atoms, optionally including heteroatoms such as oxygen, sulfur, or nitrogen. In another embodiment, the chain moieties include alkyl, alkenyl, and alkynyl moieties. In a further embodiment, the chain moiety is a fatty acid chain.

The term “fatty acid chain” includes the alkyl, alkenyl, and alkynyl chains of naturally occurring and non-naturally occurring fatty acids. The chains may be straight or branched. The fatty acid chain may be saturated or unsaturated. Examples of saturated fatty acid chains include, but are not limited to, myristic acid chains, palmitic acid chains, stearic acid chains, arachidic acid chains, behenic acid chains, lignoceric acid chains, or cerotic acid chains. Examples of unsaturated fatty acid chains include, but are not limited to, palmitoleic acid chains, olelic acid chains, vaccenic acid chains, linoleic acid chains, or arachidonic acid chains.

The term “linking moiety” includes moieties which are capable of connecting the phosphate group (PO₄⁻), and the ester groups (—OC═OR¹ and —OC═OR²), such that the phosphatidic acid compound is capable of performing its intended function, e.g., modulate apoptosis. In one embodiment, the linking moiety is alkyl, alkynyl, or alkynyl. In another embodiment, the linking moiety may be substituted with substituents which allow it to perform its intended function. In a further embodiment, the linking moiety is alkyl, e.g., n-butyl.

The term “prodrug moiety” includes moieties which may be cleaved in vivo, to yield an active compound. The prodrug moieties may be metabolized in vivo by enzymes or by other mechanisms to phosphatidic acids. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the phosphatidic acid compounds, or by separately reacting the purified phosphatidic acid compound in its free acid form with a suitable derivatizing agent. Examples of prodrug moieties include substituted and unsubstituted, branched or unbranched lower alkyl phosphatidic ester moieties, (e.g., ethyl phosphatidic esters, propyl phosphatidic esters, butyl phosphatidic esters, pentyl phosphatidic esters, cyclopentyl phosphatidic esters, hexyl phosphatidic esters, cyclohexyl phosphatidic esters), lower alkenyl phosphatidic esters, dilower alkyl-amino lower-alkyl phosphatidic esters (e.g., dimethylaminoethyl phosphatidic ester), acylamino lower alkyl phosphatidic esters, acyloxy lower alkyl phosphatidic esters (e.g., pivaloyloxymethyl phosphatidic ester), aryl phosphatidic esters (phenyl phosphatidic ester), aryl-lower alkyl phosphatidic esters (e.g., benzyl phosphatidic ester), substituted (e.g., with methyl, halo, or methoxy substituents) aryl and aryl-lower alkyl phosphatidic esters, etc.

In a further embodiment, the phosphatidic acid compound is of the formula (II):

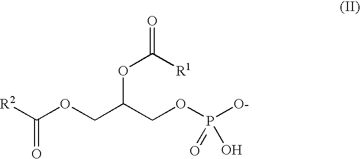

wherein

R¹ and R² are each independently selected chain moieties.

In yet another further embodiment, the phosphatidic acid compound is of the formula (III):

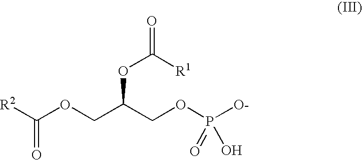

wherein

R¹ and R² are each independently selected chain moieties.

In a further embodiment, each of R¹ and R² is a fatty acid chain. In another further embodiment, the phosphatidic acid compound is L-α-phosphtidic acid (1,2 diacyl-sn-glycerol-3-phosphate) or a pharmaceutically acceptable salt thereof.

The production of phosphatidic acid compounds is known in the art and is described in Eibl, H. (1980) Chemistry and Physics of Lipids, 26:405. Phosphatidic acid compounds may also be obtained from commercial sources such as Sigma-Aldrich or Avanti Polar Lipids. Additionally, phosphatidic acid compounds may be purified from plant and animal sources. Methods of purification are described in Patton et al. (1982) J. Lipid Res. 23:190.

The term “apoptosis associated disorder” includes diseases, conditions, and disorders caused or related to apoptosis. The apoptosis associated disorder may be associated with an enhanced or increased rate of apoptosis (as compared to the rate that is desired for the particular subject), or a decreased rated of apoptosis (also as compared to the rate that is desired for the particular subject.). Examples of apoptosis associated disorders include, but are not limited to, eye disorders, neurodegenerative diseases, bone disorders (e.g. osteoarthritis), viral infection (e.g. HIV), organ (e.g., lung, heart, liver, kidney, skin, eye, etc.), tissue or cell transplantation, immunosupression, degenerative liver conditions, reperfusion damage disorders, muscle loss (e.g. muscular dystrophy, cachexia), infarction, stroke, autoimmune disorders, inflammation, myoma, muscular atrophy, glaucoma, systemic inflammation response syndrome, adult respiratory distress syndrome, cerebral malaria, chronic pneumonia, pulmonary sarcosidosis, enteris, burn damage, disorders characterized by increased protein loss, chronic renal insufficiency, ischemia, and hypertrophic disorder. In a further embodiment, the apoptosis of the apoptosis disorder is associated with the trophic insufficiency pathway, hypoxia/ischemia pathway, rotenone pathway, glutamate pathway, and/or ceramide pathway. In a further embodiment, the apoptosis associated disorder is associated with glyceraldehyde-3-phosphate dehydrogenase (GAPDH).

The term “associated with” includes downstream, upstream, and direct interactions with the particular agent or pathway and such that apoptosis is modulated. For example, a particular disease maybe associated with GAPDH. The disease associated with GAPDH may alter the concentration of GAPDH, thus modulating apoptosis and, potentially, adversely affecting the subject. It also includes disorders which can be treated by modulating GAPDH (e.g., the concentration of GAPDH) or other members of the GAPDH pathway. Examples of members of the GAPDH pathway include iGluR, JNK, c-JUN, p53, BCL-2, BCL-X, BAD, CREB, etc. Examples of apoptotic associated disorders associated with GAPDH include but are not limited to neurodegenerative disorders (e.g., Parkinson's disease, multiple sclerosis, peripheral neuropathies, etc.), cerebral hypoxia and eye disorders (e.g., glaucoma).

In a further embodiment, the invention pertains to methods of treating apoptosis associated disorders which are associated with trophic insufficiency pathway apoptosis. Trophic insufficiency apoptosis includes apoptosis which is caused by the sudden withdrawal of NGF and serum. Examples of disorders which are associated with trophic insufficiency pathway apoptosis include neurodegenerative disorders and glaucoma. Examples of agents associated with the trophic insufficiency pathway which maybe modulated include, for example, but are not limited to, SMase, ceramide, p53, GAPDH, BCL-2, BCL-X, p21, BAX, etc.

In another embodiment, the invention pertains to methods of treating apoptosis associated disorders wherein the apoptosis associated disorder is associated with ceramide pathway apoptosis. Examples of apoptosis associated disorders associated with ceramide pathway apoptosis include, but are not limited to, neurodegenerative disorders (e.g., Parkinson's disease), immune disorders (e.g., HIV), and retinitis pigmentosa. In a further embodiment, the apoptosis associated disorders of the invention do not comprise apoptosis associated disorders which are associated only with the ceramide pathway. Examples of agents associated with the ceramide pathway apoptosis include, but are not limited to, phosphatase, Akt, p53, p21, GAPDH, BCL-2, BAX, and BAD.

In another embodiment, the invention pertains to apoptosis associated disorder is associated with rotenone pathway apoptosis. Rotenone inhibits mitochondrial respiratory complex I. An example of a rotenone pathway apoptosis associated disorder is Parkinson's disease (Beterbet et al. 2000, Nature Neuroscience, 3, 12, 1301-1306).

In a further embodiment, the invention pertains to apoptosis associated disorder associated with glutamate pathway apoptosis. Examples of apoptosis associated disorders associated with glutamate pathway apoptosis include, but are not limited to, disorders relating to nerve cell death (e.g., in stroke), amyotrophic lateral sclerosis, and glaucoma. Examples of agents associated with the glutamate pathway apoptosis include, but are not limited to, iGluR, p53, GAPDH, BCL-2, BCL-X, and BAX.

The term “eye disorders” include glaucoma, proliferative vitreoretinopathy (PVR), retinal detachment, corneopathies, non-exudative age-related macular degeneration (dry AMD), exudative (wet) AMD, retinopathies (e.g., diabetic), hereditary retinal degenerations including retinitis pigmentosa (hereditary and sporadic cases), Usher's syndrome, Fundus Albipunctatus, Stargardt's Disease, retinal degenerations owing to systemic inborn errors of metabolism (e.g., Tay-Sachs, Gauchers, Hereditary Telangiectasia), retrobulbar optic neuritis, Leber's congenital amaurosis, central or branch retinal artery occlusion, central or branch vein occlusion, photoreceptor degeneration (e.g., degeneration associated with chronic macular edema, toxic retinopathies due to systemic drugs, rhegmatogenous retinal detachment, non-rhegmatogenous retinal detachment, etc.), keratocyte loss (e.g., loss associated with excimer laser keratectomy such as Lasik and PRK), loss of conjunctival cells, loss of lacrimal gland cells (e.g., loss due to severe allergic reactions such as Stevens Johnson syndrome, Sjogren's Syndrome, keratoconjunctivitis sicca, radiation therapy, etc.), loss of motor nerve function in diabetic and non-diabetic oculomotor nerve palsies, loss of visual field (e.g., loss due to ischemia, tumor pressure, and radiation-induced damage of the visual cortex of the occipital lobe, the optic radiation, the lateral geniculate, the optic tracts, chiasm, and/or optic nerve), and other diseases or disorders of the eye associated with apoptosis.

In a further embodiment, the invention pertains to a method for treating glaucoma in a subject, by administering an effective amount of a phosphatidic acid compound of the invention to the subject.

In a further embodiment, the phosphatidic acid compound is administered in combination with a known method of treating the apoptosis associated disorder.

The term “in combination with” a known method of treatment is intended to include simultaneous administration of or treatment with the phosphatidic acid compound and the known method of treatment, administration of or treatment with the phosphatidic acid compound first, followed by the known method of treatment and administration of or treatment with the known method of treatment first, followed by the phosphatidic acid compound second. Any of the therapeutically useful method known in the art for treating a particular apoptosis associated disorder can be used in the methods of the invention.

Known methods for treatment of non-exudative age-related macular degeneration (dry AMD) include the administration of luten and sub-acute diode laser treatment. Known methods of treatment of exudative (wet) AMD include laser photocoagulation and photodynamic therapy. Known methods of treating retinopathies, such as, for example diabetic retinopathy, include oral hypoglycemics and laser treatments (e.g., focal and pan-retinal laser photocoagulation). Examples of treatments for hereditary retinal degeneration, such as retinitis pigmentosa (e.g., both hereditary and sporadic cases), Usher's syndrome, Fundus Albipunctatus, and Stargardt's Disease include administering Vitamin A supplements, and potentially, gene therapies in the future. Known methods of treatment of field loss, e.g., field loss due to glaucoma, include, but are not limited to trabeculoplasty, i ridectomy, iridotomy, filtration surgery, administration of drugs that increase aqueous outflow through the trabecular meshwork or through the uveal tract, and administration of drugs that decrease aqueous production. Examples of known methods of treatment for retrobulbar optic neuritis include the administration of steroids. Known methods for treating central or branch retinal artery occlusions include the administration of anticoagulants and clot busting drugs as well as laser treatments. Central or branch vein occlusions are treated using similar methods. Photoreceptor degeneration, such as that associated with chronic macular edema, is generally treated by the administration of steroids. For the treatment of toxic retinopathies due to systemic drugs, a known method of treatment includes withdrawal of the drug. Examples of known methods of treating photoreceptor degeneration associated with rhegmatogenous retinal detachment, include repairing the detachment. Known methods for treating photoreceptor degeneration associated with non-rhegmatogenous retinal detachment, include eliminating the cause of the exudative detachment (e.g., by a subretinal neurovascular net). Methods of treating a loss of conjunctival cells or a loss of lacrimal gland cells in severe allergic reactions (e.g., Stevens Johnson syndrome) include withdrawing the drug causing the allergic reaction or by administering steroids. Known methods of treating a loss of visual field owing to ischemia, tumor pressure, or radiation-induced damage of the visual cortex of the occipital lobe, the optic radiation, the lateral genieulate, the optic tracts, chiasm, or the optic nerve, include the administration of steroids or clot busting drugs, and, when appropriate, removing tumors.

In a further embodiment, the invention pertains to methods of using phosphatidic acid compounds to treat eye disorders in a subject. The method includes administering to the subject an effective amount of a phosphatidic acid compound, e.g., a compound of formula (I), (II) or (III). In a further embodiment, the method includes the coadministration of a pharmaceutically acceptable carrier. In another further embodiment, the subject is a human, e.g., a human suffering from or at risk of suffering from an eye disorder.

The term “neurodegenerative disorders” or “neurodegenerative diseases” include neurodegenerative and other neurological disorders which are related or can be associated with apoptosis or the degeneration of neurons or neural cells. Classic examples of neurodegenerative diseases include Alzheimer's disease, Parkinson's disease, Huntington's disease, Pick's disease and amyotrophic lateral sclerosis. Other conditions in which neurons or neural cells degenerate include retinitis pigmentosa, cerebellar degeneration, progressive supranuclear palsy, Jakob-Creutzfiedlt disease, diabetic and toxic neuropathies, traumatic nerve injury, AIDS encephalitis, acute disseminated encephalomyelitis, stroke, and aging. The terms also include conditions like multiple sclerosis, wherein neurons appear to degenerate secondarily to demylination.

In a further embodiment, the invention pertains to methods of using phosphatidic acid compounds to treat neurodegenerative disorders in a subject. The method includes administering to the subject an effective amount of a phosphatidic acid compound, e.g., a compound of formula (I), (II) or (III). In a further embodiment, the method includes the coadministration of a pharmaceutically acceptable carrier. In another further embodiment, the subject is a human, e.g., a human suffering from or at risk of suffering from a neurodegenerative disorder.

In another embodiment, the invention pertains to methods treating neurodegenerative disorders by administering an effective amount of a phosphatidic acid compounds, such that the disorder is treated. Examples of phosphatidic acid compounds which may be used include those of the formulae (I), (II), and (III). In another further embodiment, the invention also pertains to methods of modulating apoptosis in neurons, glial cells, oligodendrocytes, Schwann cells, and neuronal stem cells, by administering an effective amount of a phosphatidic acid compound (e.g., a compound of formula (I), (II), or (III)). The cells may be within a subject or outside of the subject's body. The invention also pertains to methods of treating disorders which are associated with apoptosis of these or other neuronal cells.

In another embodiment, the invention pertains to modulating an apoptosis associated state which is associated with one or more apoptosis modulating agents. Examples of apoptosis modulating agents are the species shown in FIG. 1, although other agents also involved in the initiation, decision and degradation phase of apoptosis are also included. Examples of apoptosis modulating agents include agents which when the concentration, activity or presence of can modulate apoptosis in a subject.

The term “apoptotic modulating agents” includes agents which are involved in modulating (e.g., inhibiting, decreasing, increasing, promoting) apoptosis. Examples of apoptotic modulating agents include proteins which comprise a death domain such as, but not limited to, Fas, TNF RI, DR1, DR2, DR3, DR4, DR5, DR6, FADD, and RIP. Other examples of apoptotic modulating agents include, but are not limited to, TNF alpha, Fas ligand, TRAIL, bcl-2, p53, BAX, BAD, Akt, CAD, PI3 kinase, PP1, and caspase proteins.

Apoptotic modulating agents may be soluble or membrane bound (e.g. receptor or ligand). The term “bone disorders” include disorders of the bone which are associated with or affected by apoptosis. In one embodiment, bone disorders include disorders associated with enhanced bone formation. Enhanced bone formation may occur, for example, when osteoblasts are inhibited from entering apoptosis. In other embodiments, the bone disorders may be associated with decreased or low bone formation rate. Examples of such disorders include, but are not limited, bone breaks, osteoporosis, and osteoarthritis.

The term “reperfusion damage disorders” includes disorders associated with a decrease in blood flow causing hypoxia, and subsequent reperfusion of blood to the area. Examples of such disorders include, but are not limited to, artery obstruction, myocardial infarction, cerebral infarction, spinal/head trauma, and frostbite. Apoptosis can occur upon reperfusion of blood to the affected area. Damage can occur, for example, to the heart, brain, kidney, liver, spleen, lung or testes. In one embodiment, the invention pertains to methods of treating reperfusion damage in a subject, by administering an effective amount of a phosphatidic acid compound of the invention (e.g., a compound of formula (I), (II) or (III). In a further embodiment, the invention pertains to methods of treating artery obstruction, myocardial infarction, cerebral infarction, spinal/head trauma, or frostbite, by administering an effective amount of a compound of formula (I), (II), or (III).

Degenerative conditions of the liver are also included as apoptosis associated disorders. Acetaminophen, cocaine, ethanol, hepatitis and endotoxin have been shown to induce apoptosis in hepatocytes. Thus, in one embodiment, the invention pertains to methods of treating liver degenerative conditions, by administering an effective amount of a phosphatidic acid compound of the invention (e.g., a compound of formula (I), (II), or (III)). In another embodiment, the invention pertains to methods of treating liver degenerative conditions, by administering an effective amount of a phosphatidic acid compound of the invention to decrease apoptosis (e.g., in liver cells, such as, for example, hepatocytes).

Muscle disorders associated with apoptosis are also included as apoptosis associated disorders. Examples of such disorders include, but are not limited to muscular dystrophy. In one embodiment, the invention pertains to methods of modulating (e.g., decreasing or increasing) apoptosis in muscle cells in a subject, by administering an effective amount of a phosphatidic acid compounds (e.g., a compound of formula (I), (II), or (III)). In a further embodiment, the invention pertains to methods of using phosphatidic acid compounds to decrease apoptosis of muscle cells. Examples of disorders which are associated with decreases in muscle cells include muscle atrophy, muscular dystrophy and cachexia.

In one embodiment, the invention pertains to methods of using phosphatidic acid compounds to modulate apoptosis to treat stroke, autoimmune disorders, inflammation, myoma, muscular atrophy, systemic inflammation response syndrome, adult respiratory distress syndrome, cerebral malaria, chronic pneumonia, pulmonary sarcosidosis, enteris, burn damage, disorders with increased protein loss, chronic renal insufficiency, ischemia, or hypertrophic disorders.

In another embodiment, the invention pertains to methods of enhancing bone formation by decreasing apoptosis of bone cells in a subject, by administering an effective amount of a phosphatidic acid compound of formula (I), (II), or (III). For example, the phosphatidic acid compounds of this invention can be used to enhance bone formation to promote the reformation of a bone break, or treat osteoporosis or osteoarthritis.

Immunosupression, such as caused by HIV, chemotherapy, radiation or immunosuppressive drug therapy, can trigger apoptosis in a variety of cell types. For example, chemotherapy can induce apoptosis in the digestive tract. In one embodiment, the invention pertains to methods of using phosphatidic acid compounds to modulate apoptosis caused by immunosuppressive agents, by administering an effective amount of a phosphatidic acid compound of the invention (e.g., a compound of formula (I), (II), or (III)). Examples of agents that may cause immunosuppression include, but are not limited to, HIV, chemotherapy, radiation and immunosuppressive drug therapy.

In a further embodiment, the apoptosis associated disorder is a viral infection. Viral infections may cause high levels of apoptosis in infected cells. For example, the HIV virus can produce a high level of apoptosis in CD4+ T cells (Thompson, C. (1995) Trends Cell Bio. 5:27). In one embodiment, the invention pertains to methods of treating a viral infection in a subject, by administering to the subject an effective amount of a phosphatidic acid compounds (e.g., a compound of formula (I), (II), or (III).

In another embodiment, the invention pertains to methods of decreasing apoptosis of cells during a viral infection in a subject, by administering to the subject an effective amount of a phosphatidic acid compound of the invention (e.g., a compound of formula (I), (II), or (III)), such that apoptosis is decreased. In a further embodiment, the viral infection is a retroviral infection (e.g., HIV) or a viral infection of the immune system. In another embodiment, the cells are CD4+ T cells.

The term “subject” includes organisms capable of suffering from an apoptosis associated disorder, such as mammals (e.g. primates (e.g., monkeys, gorillas, chimpanzees, and, advantageously, humans), goats, cattle, horses, sheep, dogs, cats, mice, rats, rabbits, pigs, dolphins, ferrets, squirrels), reptiles, or fish, and transgenic species thereof. In one embodiment, the subject is suffering from or at risk of suffering from an apoptosis associated disorder. The term subject is intended to include living organisms in which apoptosis can occur, e.g., mammals. The term also includes parts of the above discussed organisms which may or may not be attached to said organism. For example, the term includes organs which have been removed from one subject for transplant into another.

The term “therapeutically effective amount” or “effective amount” includes an amount of the compound which is effective in treating an apoptosis associated disorder. A therapeutically effective amount may be readily determined by an attending diagnostician, as one skilled in the art, by the use of known techniques and by observing results obtained under analogous circumstances.

The term “treated,” “treating” or “treatment” includes the diminishment or alleviation of at least one symptom associated or caused by apoptosis associated disorder being treated. For example, treatment can be diminishment of one or several symptoms of a disorder or complete eradication of a disorder.

Cells cultured in vitro often have a limited life span. Limiting apoptosis of cells in vitro may be useful to extend cell life. Genetically engineered cell lines often die in culture after a certain level of protein production. Inhibition of apoptosis could increase the life span of these cell lines and result in higher protein production. For example, a cell line engineered to produce a protein will eventually die in culture conditions after a certain level of protein expression. Thus, in one embodiment, the invention pertains to methods of modulating (e.g., decreasing or increasing) apoptosis of a cell in vitro, by administering an effective amount of a compound of formula (I), (II), or (III). In a further embodiment, the invention pertains to methods of using phosphatidic acid compounds to modulate apoptosis in a genetically engineered cell.

In one embodiment, the invention pertains to methods of maintaining a biological sample ex vivo, such as, for example, organs, tissues and cells, e.g., for transplantation. The method includes contacting the sample a phosphatidic acid compound of the invention, such that the organ, tissue or cell is maintained, e.g., by inhibiting apoptosis. Examples of phosphatidic acid compounds which may be used include compounds of formula (I), (II), and (III). Prior to, during, and after transplantation, a number of cells may enter apoptosis thereby decreasing the likelihood of successful organ transplantation. In an embodiment, the invention pertains to a method for modulating, e.g., decreasing, the rate of apoptosis of cells in an organ during and after transplantation in a host. Examples of organs which may be transplanted in hearts, lungs, kidneys, skin, liver, etc.

The term “genetically engineered cell” includes cells in which production of a specific DNA molecule, RNA molecule, or protein is encouraged or promoted. For example, a chinese hamster ovary (CHO) cell line could be transfected with a DNA sequence containing the DNA for a protein and an appropriate promoter. Induction of the promoter can result in production of the desired protein.

Phosphatidic acid compounds of this invention may be used for any apoptosis associated disorder. There are a variety of methods to test for apoptosis. These methods can be used to determine in what disease states or conditions apoptosis occurs or to determine the effectiveness of the phosphatidic acid compounds of this invention. One method of evaluating the ability of a phosphatidic acid compound to inhibit apoptosis entails inducing apoptosis in cells (with an agent such as rotenone glutamate, actinomycin D, ceramide or TNF alpha) in the presence and absence of the phosphatidic acid compounds.

Apoptosis can then be implicated in cell death by a variety of techniques known in the art. For example, DNA ladders can be used with DNA gel electrophoresis to show characteristic DNA cleavage patterns (Herman et al. (1994) Nucleic Acid Research 22:5506). Another technique is the in situ end-labeling (ISEL) of cut DNA. Apoptosis can also be identified by demonstrating nuclear chromatin condensation by using florescent DNA binding dyes like YOYO-1. Furthermore, general or specific caspase inhibitors can be used to demonstrate cysteine protease/caspase dependency. The decrease of mitochondrial membrane potential can also be studied as an indicator and can be visualized using mitochondrial potentiometric dye fluorescence images with epifloresence microscopy or laser confocal scanning microscopy.

The phosphatidic acid compounds of this invention can be incorporated into pharmaceutical compositions suitable for administration. Such compositions typically comprise the phosphatidic acid molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g. the phosphatidic acid compounds) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are, generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the phosphatidic acid compounds are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc.

Liposomal suspensions (including liposomes targeted to specific cells with antibodies to specific antigens) can also be used as pharmaceutically acceptable carriers. These can be prepared according to methods known to those skilled in the art, for example, as described in U.S. Pat. No. 4,522,811. Lipid based delivery systems have the advantage of being able to deliver hydrophobic drugs. Another delivery system for hydrophobic drugs is a cochleate delivery system from BioDelivery Sciences International, as described in U.S. Pat. No. 6,153,217.

Referred to as the PHOTOTARGET® system, light-targeted delivery of drugs and/or diagnostic imaging dyes to the vasculature of the retina is a potential delivery mechanism for phosphatidic acid compounds of the invention. The method includes intravenous administration of a liposome vesicles which comprise artificial phospholipids encapsulating a drug or dye. A short, low-intensity pulse of light delivered warming of the target tissue, (retinal or choroidal blood vessels) thereby thermally rupturing the liposomes and releasing a small bolus of drug or dye from circulating liposomes. The intensity of the light alone is insufficient to damage either the targeted or the surrounding tissues (See, for example, U.S. Pat. No. 6,248,727; U.S. Pat. No. 6,140,314; U.S. Pat. No. 5,935,942; U.S. Pat. No. 4,891,043).

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The phosphatidic acid compounds of this invention are administered to subjects in a biologically compatible form suitable for pharmaceutical administration in vivo to modulate, e.g., inhibit, apoptosis. By “biologically compatible form suitable for administration in vivo” is meant a form of the molecule to be administered in which any toxic effects are outweighed by the therapeutic effects of the protein. Administration of an agent as described herein can be in any pharmacological form including a therapeutically active amount of an agent alone or in combination with a pharmaceutically acceptable carrier.

The phosphatidic acid compounds of the present invention may contain one or more acidic functional groups and, thus, are capable of forming pharmaceutically acceptable salts with pharmaceutically acceptable bases. The term “pharmaceutically acceptable salts” in these instances includes relatively non-toxic, inorganic and organic base addition salts of compounds of the present invention. These salts can be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “pharmaceutically acceptable esters” refers to the relatively non-toxic, esterified products of the phosphatidic acid compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl and propyl esters. (See, for example, Berge et al., supra.) A preferred ester group is an acetomethoxy ester group.

The pharmaceutical compositions can be included in a container, pack, or dispenser together with instructions for administration, e.g., to treat an apoptosis associated disorder.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer. Likewise, preferred cycloalkyls have from 4-7 carbon atoms in their ring structure, and more preferably have 5 or 6 carbons in the ring structure.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)). The term “alkyl” also includes the side chains of natural and unnatural amino acids. Examples of halogenated alkyl groups include fluoromethyl, difluoromethyl, trifluoromethyl, chloromethyl, dichloromethyl, trichloromethyl, perfluoromethyl, perchloromethyl, perfluoroethyl, perchloroethyl, etc.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, imidazoline, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, isoindole, indan or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

Those skilled in the art will recognize, or be able to ascertain using no more than routine experimentation, numerous equivalents to the specific procedures described herein. Such equivalents are considered to be within the scope of the present invention and are covered by the claims.

This invention is further illustrated by the following examples which should not be construed as limiting. The contents of all references, patents and published patent applications cited throughout this application, as well as the Figures, are incorporated herein by reference.

### EXEMPLIFICATION OF THE INVENTION

### Example 1

### Reduction of Apoptosis Initiated by NGF and Serum Withdrawal, Ceramide, Rotenone, and Glutamate

Phospholipase D2 (PLD2) converts phosphatidyl choline to phosphatidic acid (PA) and choline. Glydceraldehyde-3-phosphate dehydrogenase (GAPDH) upregulation is important to some neuronal apoptosis. Co-immunoprecipitation for multiply transfected cells and those in early apoptosis showed that PLD2 and GAPDH, both normally cytosolic, colocalize densely in the nucleus in apoptotic cells. PA was shown to reduce apoptosis initiated by ceramide, possibly by preventing protein kinase B (PKB/Akt) dephosphorylation by a ceramide activated phosphatase. It was hypothesized that GAPDH/PLD2 binding may reduce PA levels and render neurons vulnerable to apoptosis by opposing phosphoinosital-3-kinase (PI3K) induced Akt phosphorylation. In a concentration dependent manner, 2 to 30 μM PA reduced apoptosis initiated by NGF and serum withdrawal, ceramide, rotenone, or glutamate, but not by peroxide or atractyloside. PA reduced the mitochondrial membrane potential dissipation that occurs early in some forms of apoptosis signaling and may indicate increased mitochondrial membrane permeability. Pharmacological inhibition of PI3K reduced PA anti-apoptosis and PA maintenance of mitochondrial membrane potential. PA therefore can reduce apoptosis other than ceramide apoptosis, in part by altering the balance between Akt phosphorylation and dephosphorylation.

Culture Of NGF Differentiated PC12 Cells.

PC12 cells (ATCC, Manassas, Md.) were propagated in minimum essential medium (MEM) containing 10% horse serum, 5% fetal bovine serum, 2 mM L-glutamine, 50 units/ml penicillin, and 50 μg/ml streptomycin (MEM with serum, MIS), all purchased from Life Technologies (Rockville, Md.). The cells were grown on 24 well plates (8×10⁴ cells/well) for counting of intact nuclei as an estimate of survival, poly-L-lysine treated coverslips (1×10⁴ cells/coverslip) for imaging with epiflorescence microscopy or laser confocal scanning microscopy (LCSM) or 100 mm dishes (1×10⁶ cells/plate) for protein chemistry. The cells were differentiated for 6 days in M/S supplemented with 100 ng/ml 7S NGF (Upstate Biotech., Lake Placid, N.Y.). MEM with serum and NGF is abbreviated as M/S+N (see (Tatton et al., 1994, J. Neurochemistry, 63:1572-1575; Wadia et al., J. Neuroscience, 1998, 18: 932-947; Carlile et al., 2000, Molecular Pharmacology, 57:2-12) for further details of culture and treatment).

Initiation Of Apoptosis In NGF Differentiated PC12 Cells

Serum and NGF Withdrawal: Following incubation for 6 days in MEM with serum and NGF, cells underwent three successive washes in Hanks' Balanced Salt Solution (HBSS; Life Technologies, Rockville, Md.) to remove NGF and serum-borne trophic agents and then were replaced into MEM with serum and NGF for controls or into MEM without serum and NGF to induce apoptosis by serum and NGF withdrawal.

C2-Ceramide, Rotenone, Peroxide, and Atractyloside Exposure: After 6 days of exposure to serum and NGF, cultures maintained in MEM with serum and NGF were treated for 24 hours with either vehicle (HBSS) as a control, or concentrations of C2-ceramide in HBSS varying from 2 to 50 μM, concentrations of rotenone in BSS varying from 2 to 50 nM, concentrations of H₂O₂ in HBSS varying from 0.01 to 0.25 mM H₂O₂ or concentrations of atractyloside in HBSS varying from 2 to 20 mM.

Culture Of Cerebellar Granule Neurons.

Cerebellar granule neurons were obtained from postnatal day 7 rat pups by enzymatic digestion and maintained in serum supplemented Eagle's Basal medium. Viable cells were plated at a density of 500,000 cells/ml onto poly-L-lysine coated tissue culture plastic or glass coverslips. Cytosine arabinoside (Ara-C) (10 μM) was added at 24 hours to halt glial proliferation. To induce apoptosis in the cerebellar granule neurons, 10⁻⁴ to 10⁻⁶ M glutamate was added on day 7 to the culture.

Estimation of Survival And Levels Of Apoptosis In NGF Differentiated PC12 Cells And Cerebellar granule Neurons.

Both cell survival and the percentages of cells with evidence of apoptotic nuclear degradation were assessed for all treatments. To estimate survival, the cells were seeded at a density of 8×10⁴ cells/well in 24 well plates. Cells were harvested 24 hours after treatment and lysed. Intact nuclei were counted using a hemocytometer (see see (Tatton et al., 1994, J. Neurochemistry, 63:1572-1575; Wadia et al., J. Neuroscience, 1998, 18: 932-947; Carlile et al., 2000, Molecular Pharmacology, 57:2-12) for details of treatment and counting methods).

Percentages of cells with apoptotic nuclei were determined for cells grown on poly-L-lysine treated coverslips (density 1×10⁴/coverslip). At varying times after treatment the cells were stained with the DNA binding dye YOYO-1 (Molecular Probes, Eugene, Oreg.) to reveal chromatin condensation as a marker of apoptotic nuclear degradation see (Tatton et al., 1994, J. Neurochemistry, 63:1572-1575; Wadia et al., J. Neuroscience, 1998, 18: 932-947; Carlile et al., 2000, Molecular Pharmacology, 57:2-12). Cells on coverslips were washed three times in PBS followed by 100% methanol incubation at −20° C. for 30 seconds. The methanol was then replaced with YOYO-1 (1.5 μM in PBS) for thirty minutes at room temperature. After three PBS washes, the cells on coverslips were mounted in Aquamount Gurr (EM Industries, Cincinnati Ohio) for LCSM imaging. The total number of YOYO-1 stained nuclei with chromatin condensation were counted on twenty-five 40× fields for each coverslip, each field chosen by pairs of randomly-generated x-y coordinates. The proportion of nuclei with chromatin condensation were expressed as a percentage of the total number of cells in each field. The values were pooled for three coverslips for each treatment and time point.

Measurement Of Mitochondrial Membrane Potential (ψ_{M})

Live cell Δψ_{M} measurement: Cells on polylysine coated coverglass were incubated with 100 nM tetramethylrodaminemethylester (TMRM, Molecular probes, Eugene, Oreg.) or 10 μg/ml JC-1 (Molecular Probes, Eugene, Oreg.) to provide an estimate of Δψ_{M}. The coverslips were transferred to a gas and temperature controlled circulating live cell chamber (Medical Systems Corp., Greenvale, N.Y.). A thermocoupler immersed in the medium maintained the media temperature at 37±0.1° C. TMRM, JC-1 and chloromethyltetramethylrosamine (CMTMR, Molecular probes, Eugene, Oreg.) and JC-1 stained cells were imaged 10 minutes following dye addition and images were collected every 3 minutes for approximately 20 minutes.

Fixed cell Δψ_{M} Measurement. After treatment of NGF differentiated PC12 cells on poly-L-lysine coated 12 mm coverslips, the fixable potentiometric dye, CMTMR, was added at 137 nM for 15 minutes at 37° C. and simultaneous immunocytochemistry was performed. The cells were then rinsed once with warm Dulbecco's PBS prior to overnight fixation with ice cold 4% paraformaldehyde in PB and mounted onto slides with the aqueous mounting medium, Aquamount (Gallard-Schlesinger Ind., Garden City, N.Y.).

Florescence Microscopy. Images were obtained using a Leica TCS4D confocal microscope coupled to an argon-krypton laser or a standard epi-floresence microscope. The images were scanned or imaged using an oil immersion 100×1.4 N.A. objective to minimize focal depth. CMTMR and TMRM treated cells were imaged using 568 nm excitation and detected with 600/30 nm band pass filter. The JC-1 incubated cells were imaged using 488 nm excitation and detected using 530/30 nm band pass and 590 nm long pass filters observed by separate detectors. Image resolution was 512×512×8 bits per pixel. Grey scale images were saved in tagged image file format and transferred to a Pentium III PC running Northern Eclipse 5.0 (Empix, Imaging Ltd., Mississauga, Ontario) to measure individual mitochondria intensity according to a gray pixel scale of 0-255. To measure the intensity of mitochondrial potentiometric dye fluorescence, pixel intensity measurements from 20-40 mitochondria from each of 50-70 cells per treatment, per experiment were obtained by placing a 3×3 pixel box at 2 points in each mitochondrion. The fluorescence intensity values were automatically exported to an Excel spreadsheet for subsequent analysis in MicroCal Origin (Northampton, Mass.). For paired JC-1 images, similar boxes are placed on mitochondria as above on the 590 nm image. Once all of the boxes were placed, a mask of the distribution of boxes was copied to the corresponding 527 nm image to obtain paired measurements.

The capacity of phosphatidic acid to increase survival, decrease apoptosis and maintain mitochondrial membrane potential was determined by counting intact nuclei as an indicator of cell survival, determining the percentages of cells with nuclear chromatin (DNA) condensation by staining cells with YOYO-1, a nucleic acid binding dye, and measuring mitochondrial potentiometric dye fluorescence by imaging them with florescence microscopy.

These measurements of cell survival were used as a means to determine particular concentration ranges for different agents or insults that induce nerve cell death. It was then determined whether decreased survival resulted from apoptosis. If the decreased survival was a result of apoptosis, then it was determined whether the apoptosis involved mitochondria by Δψ_{M} measurement and/or caspases by caspase inhibitors.

Phosphatidic acid was shown to increase cell survival when the apoptosis initiating insult was ceramide, rotenone, serum and NGF withdrawal, or glutamate. The results of the experiments are summarized in Table 1 below.

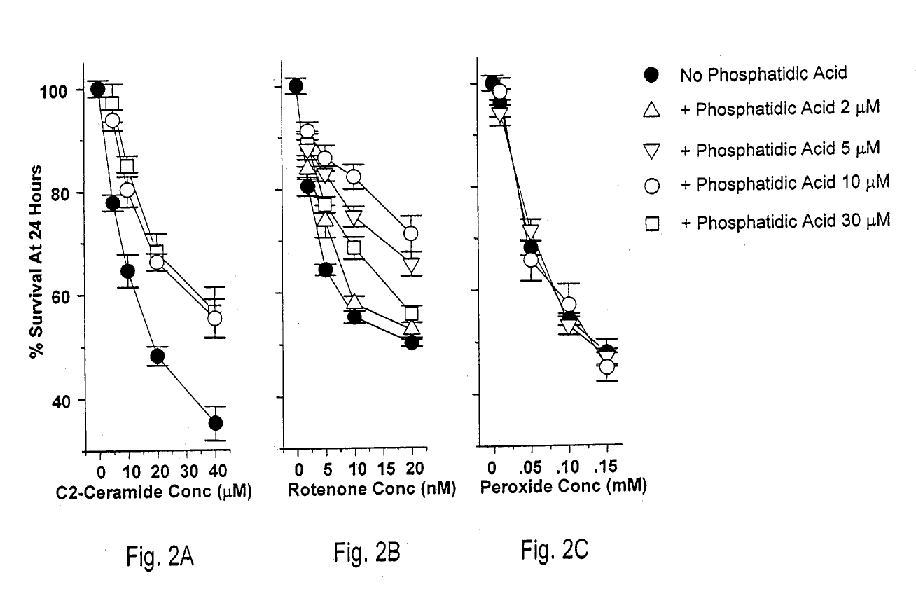

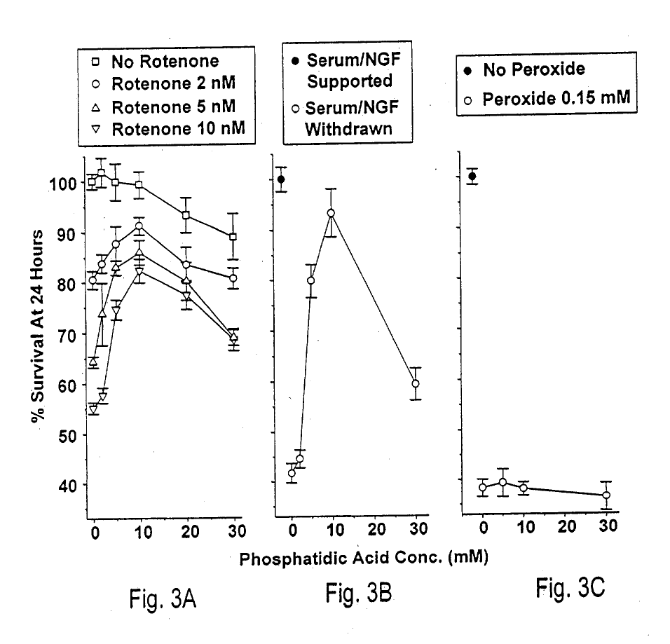

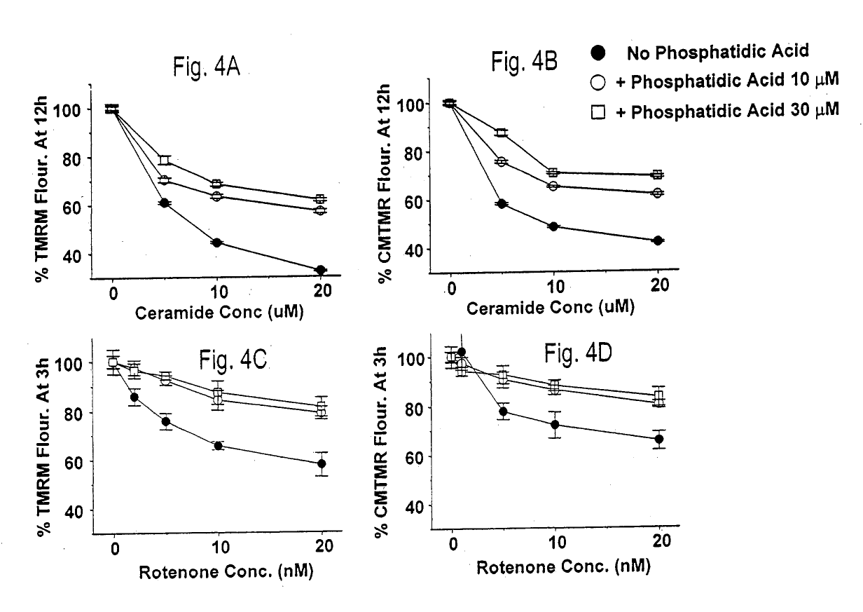

TABLE 1

| | | | PA Reduces |
|---|---|---|---|
| | | PA Decreases | Decreases In |
| Apoptosis | PA Increases | Nuclei With | Mitochondrial |
| Initiating | Survival (see | Chromatin | Membrane |
| Insult | attached figure #) | Condensation | Potential |
| Ceramide | Yes - FIG. 2A | Yes | Yes - FIG. 4A |
| | | | and 4B |
| Rotenone | Yes - FIG. 2B and | Yes | Yes - FIG. 4C |
| | 3A | | and 4D |
| Serum and | Yes - FIG. 3B | Yes | Yes |
| NGF Withdrawal | | | |
| Glutamate | Yes | Yes | nd |
| Peroxide | No - FIG. 2C and | nd | nd |
| | 3C | | |
| Atractyloside | No | nd | nd |

FIGS. 2A, 2B, and 2C show the survival of NGF differentiated PC 12 cells treated with concentrations of phosphatidic acid (ranging from 2 to 30 μM) after exposure to varying concentrations of C2-ceramide (FIG. 2A), rotenone (FIG. 2B) and peroxide (FIG. 2C). FIGS. 2A, 2B, and 2C illustrate that phosphatidic acid is effective in increasing survival after C2-ceramide (FIG. 2A) and rotenone (FIG. 2B) exposure at a concentration of 10 mM but does not alter survival after H₂O₂ exposure at any concentration (FIG. 2C). Phosphatidic acid is at least as effective with rotenone-induced death as with C2 ceramide induced death. This is unexpected since phosphatidic acid was thought to only act on survival by inhibiting a ceramide activated phosphatase and therefore opens the door to a number of apoptosis dependent diseases including Parkinson's Disease and stroke.

FIGS. 3A, 3B and 3C illustrate the increased survival induced by phosphatidic acid in rotenone exposed (FIG. 3A) and serum and NGF withdrawn (FIG. 3B) NGF differentiated PC12 cells. FIGS. 3A, 3B, and 3C also illustrate that the survival rate is dependent on the phosphatidic acid concentration. Similar to FIGS. 2A and 2B, FIGS. 3A and 3B show that phosphatidic acid is most effective at 10 mM. FIG. 3C shows that varying concentrations of phosphatidic acid does not substantially increase NGF differentiated PC12 cell survival after exposure to 0.1 mM H₂O₂ which kills about 60% of the cells.

FIGS. 4A, 4B, 4C, and 4D show that phosphatidic acid reduces the apoptosis-associated decrease of mitochondrial membrane potential (ATM) for both apoptosis initiated by ceramide (FIGS. 4A and 4B) and rotenone (FIG. 4C and FIG. 4D). Two different mitochondrial potentiometric dyes, TMRM and CMTMR, were employed in the studies and provided similar results. FIGS. 4A and 4C show results using TMRM and FIGS. 4B and 4D show the results using the dye CMTMR (see Wadia et al, 1998, J. Neuroscience 18: 932-947 for details of methods and analysis). The findings suggest that phosphatidic acid reduces increases in mitochondrial membrane permeability that are key to a number of forms of apoptosis signaling and that the action is not limited to ceramide initiated apoptosis.

1. A method of treating an apoptosis associated disorder in a subject, comprising administering a therapeutically effective amount of a phosphatidic acid compound, such that said apoptosis associated disorder is treated, wherein said phosphatidic acid compound is of formula (I): 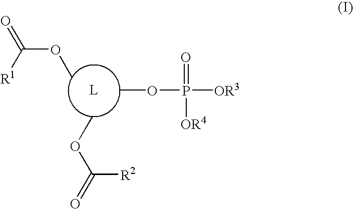
wherein
R¹ and R² are each independently selected chain moieties;
R³; and R⁴ are each independently hydrogen, absent, or a prodrug moiety;
L is a linking moiety, and pharmaceutically acceptable salts thereof.

2. The method of claim 1, wherein said phosphatidic acid compound is of the formula (II):

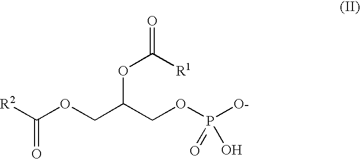

3. The method of claim 2, wherein said phosphatidic acid compound is of the formula (III):

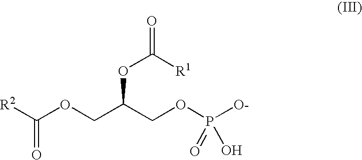

4. The method of claim 1, wherein R¹ is a chain of six to twenty atoms.

5. The method of claim 4, wherein R¹ is a fatty acid chain.

6. The method of claim 5, wherein R¹ is saturated.

7. The method of claim 6, wherein R¹ is a myristic acid chain, a palmitic acid chain, a stearic acid chain, an arachidic acid chain, a behenic acid chain, a lignoceric acid chain, or a cerotic acid chain.

8. The method of claim 5, wherein R¹ is unsaturated.

9. The method of claim 8, wherein R¹ is a palmitoleic acid chain, an olelic acid chain, a vaccenic acid chain, a linoleic acid chain, or an arachidonic acid chain.

10. The method of claim 1, wherein R² is a chain of six to twenty atoms.

11. The method of claim 10, wherein R² is a fatty acid chain.

12. The method of claim 11, wherein R² is saturated.

13. The method of claim 12, wherein R² is a myristic acid chain, a palmitic acid chain, a stearic acid chain, an arachidic acid chain, a behenic acid chain, a lignoceric acid chain, or a cerotic acid chain.

14. The method of claim 11, wherein R² is unsaturated.

15. The method of claim 14, wherein R² is a palmitoleic acid chain, an olelic acid chain, a vaccenic acid chain, a linoleic acid chain, or an arachidonic acid chain.

16. The method of claim 1, wherein said apoptosis associated disorder is associated with GAPDH.

17. The method of claim 1, wherein said apoptosis associated disorder is associated with trophic insufficiency pathway apoptosis.

18. The method of claim 17, wherein said apoptosis associated disorder is a neurodegenerative disorder or glaucoma.

19. The method of claim 1, wherein said apoptosis associated disorder is associated with ceramide pathway apoptosis.

20. The method of claim 19, wherein said apoptosis associated disorder is a neurodegenerative disorder, an immune disorder, or retinitis pigmentosa.

21. The method of claim 20, wherein said apoptosis associated disorder is Parkinson's disease.

22. The method of claim 1, wherein said apoptosis associated disorder is associated with rotenone pathway apoptosis.

23. The method of claim 22, wherein said apoptosis associated disorder is Parkinson's disease.

24. The method of claim 1, wherein said apoptosis associated disorder is associated with glutamate pathway apoptosis.

25. The method of claim 24, wherein said apoptosis associated disorder is associated with nerve cell death.

26. The method of claim 24, wherein said apoptosis associated disorder is stroke, amytrophic lateral sclerosis, or glaucoma.

27. The method of claim 1, wherein said apoptosis associated disorder is a neurodegenerative disease.

28. The method of claim 27, wherein said neurodegenerative disease occurs in neurons, glial cells, oligodendrocytes, Schwann cells, or neuronal stem cells.

29. The method of claim 27, wherein said neurodegenerative disease is Alzheimer's disease, Huntington's disease, Pick's disease, amyotrophic lateral sclerosis, retinitis pigmentosa, cerebellar degeneration, peripheral neuropathy, progressive supranuclear palsy, or Jakob-Creutzfiedlt disease.

30. The method of claim 27, wherein said neurodegenerative disease is Parkinson's disease.

31. The method of claim 1, wherein said apoptosis associated disorder is an eye disorder.

32. The method of claim 31, wherein said eye disorder is selected from the group consisting of non-exudative age-related macular degeneration, exudative age-related macular degeneration, retinopathy, retinal degeneration, retinitis pigmentosa, Usher's syndrome, fundus albipunctatus, Stargardt's disease, Tay-Sachs, Gauchers, hereditary telangiectasia, glaucoma, retrobulbar optic neuritis, Leber's congenital amaurosis, central or branch retinal artery occlusion, central or branch vein occlusion, photoreceptor degeneration, keratocyte loss, loss of conjunctival cells, lacrimal gland cells, Stevens Johnson syndrome, Sjogren's Syndrome, keratoconjunctivitis sicca, loss of motor nerve function, or loss of visual field.

33. The method of claim 1, wherein said apoptosis associated disorder is a bone disorder, a viral disorder, transplantation, immunosuppression, degenerative liver condition, reperfusion damage disorder, artery obstruction, myocardial infarction, cerebral infarction, spinal trauma, head trauma, frostbite, muscle loss, muscular dystrophy, infarction, stroke, an autoimmune disorder, inflammation, myoma, muscular atrophy, systemic inflammation response syndrome, adult respiratory distress syndrome, cerebral malaria, chronic pneumonia, pulmonary sarcosidosis, enteris, burn damage, a disorder with increased protein loss, chronic renal insufficiency, ischemia, or a hypertrophic disorder.

34. The method of claim 1, wherein said subject is a mammal.

35. The method of claim 34, wherein said mammal is a human.

36. The method of claim 1, wherein said subject is suffering from said apoptosis associated disorder.

37. The method of claim 1, further comprising administering a pharmaceutically acceptable carrier.

38. The method of claim 37, wherein said pharmaceutically acceptable carrier is acceptable for administration directly to the nervous system or to the cerebrospinal fluid.

39. The method of claim 1, further comprising administering the phosphatidic acid compound in combination with another method of treatment of said apoptosis associated disorder.

40. A method of modulating apoptosis in a cell in vitro comprising administering an effective amount of a phosphatidic acid compound such that apoptosis is modulated, wherein said phosphatidic acid compound is of formula (I): 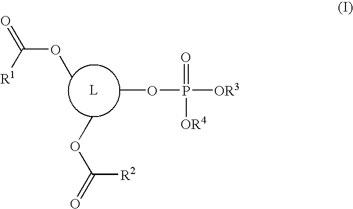
wherein
R¹ and R² are each independently selected chain moieties;
R³ and R⁴ are each independently hydrogen, absent, or a prodrug moiety;
L is a linking moiety, and pharmaceutically acceptable salts thereof.

41. The method of claim 40, wherein said cell is a genetically engineered cell.

42. A packaged pharmaceutical composition comprising:
a phosphatidic acid compound, or a pharmaceutically acceptable salt thereof, and
instructions for the use of said compound for the treatment of a apoptosis associated state, wherein said phosphatidic acid compound is of the formula (I): 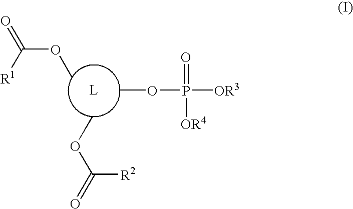
wherein
R¹ and R² are each independently selected chain moieties;
R³ and R⁴ are each independently hydrogen, absent, or a prodrug moiety;
L is a linking moiety, and pharmaceutically acceptable salts thereof.

43. A method for treating a neurodegenerative disorder in a subject, comprising administering to said subject an effective amount of a phosphatidic acid compound, such that the neurodegenerative disorder is treated, wherein said phosphatidic acid compound is of the formula (I): 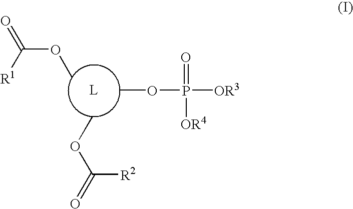
wherein
R¹ and R² are each independently selected chain moieties;
R³ and R⁴ are each independently hydrogen, absent, or a prodrug moiety;
L is a linking moiety, and pharmaceutically acceptable salts thereof.

44. The method of claim 43, wherein said neurodegenerative disorder is selected from the group consisting of Alzheimer's disease, Huntington's disease, Pick's disease, amyotrophic lateral sclerosis, retinitis pigmentosa, cerebellar degeneration, peripheral neuropathy, progressive supranuclear palsy, or Jakob-Creutzfiedlt disease

45. The method of claim 43, wherein said neurodegenerative disease is Parkinson's disease.

46. The method of claim 43, wherein said neurodegenerative disease is multiple sclerosis.

47. The method of claim 43, wherein said subject is a human.

48. The method of claim 43, wherein said subject is suffering from a neurodegenerative disorder.

49. The method of claim 43, further comprising administering a pharmaceutically acceptable carrier.

50. A method for treating an eye disorder in a subject, comprising administering to said subject an effective amount of a phosphatidic acid compound, such that the eye disorder is treated, wherein said phosphatidic acid compound is of the formula (I): 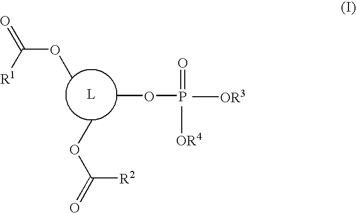
wherein
R¹ and R² are each independently selected chain moieties;
R³ and R⁴ are each independently hydrogen, absent, or a prodrug moiety;
L is a linking moiety, and pharmaceutically acceptable salts thereof.

51. The method of claim 50, further comprising administering the phosphatidic acid compound in combination with a supplementary method for treating the eye disorder.

52. The method of claim 50, wherein said eye disorder is glaucoma.

53. The method of claim 52, wherein said supplementary method is trabeculoplasty, iridectomy, iridotomy, filtration surgery, or known glaucoma treatment agents.

54. The method of claim 50, wherein said eye disorder is non-exudative age-related macular degeneration.

55. The method of claim 54, wherein said supplementary method is luten or sub-acute diode laser treatment.

56. The method of claim 50, wherein said eye disorder is exudative age-related macular degeneration.

57. The method of claim 56, wherein said supplementary method is laser photocoagulation or photodynamic therapy.

58. The method of claim 50, wherein said eye disorder is retinopathy.

59. The method of claim 58, wherein said supplementary method is administering hypoglycemics, laser treatment or withdrawing toxic drugs.

60. The method of claim 50, wherein said eye disorder is retinitis pigmentosa, Usher's syndrome, fundus albipunctatus, or Stargardt's disease.

61. The method of claim 60, wherein said supplementary method is administering vitamin A or nucleic acids.

62. The method of claim 50, wherein said eye disorder is Tay-Sachs, keratocyte loss, lacrimal gland cell loss, oculomotor nerve palsy, Gauchers, Leber's Congenital Amaurosis or hereditary telangiectasia.

63. The method of claim 50, wherein said eye disorder is retrobulbar optic neuritis or photoreceptor degeneration.

64. The method of claim 63, wherein said supplementary method is administering steroids.

65. The method of claim 50, wherein said eye disorder is a central or branch retinal artery or vein occlusion.

66. The method of claim 65, wherein said supplementary method is laser treatment or the administering anticoagulants or clot busters.

67. The method of claim 50, wherein said eye disorder is photoreceptor degeneration.

68. The method of claim 67, wherein said supplementary treatment is repairing the detachment or treating the cause of the detachment.

69. The method of claim 50, wherein said eye disorder is loss of conjunctival cells or lacrimal gland cells.

70. The method of claim 69, wherein said supplementary treatment comprises administering steroids.

71. The method of claim 50, wherein said eye disorder is loss of visual field.

72. The method of claim 71, wherein said supplementary treatment comprises administration of steroids or clot busting drugs.

73. The method of claim 50, wherein said subject is a human.

74. A method for treating an apoptosis associated disorder in a subject, comprising administering to said subject an effective amount of a phosphatidic acid compound, such that the mitochondrial membrane potential is maintained, wherein said phosphatidic acid compound is of the formula (I): 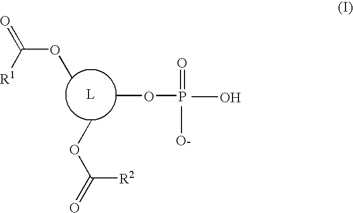
wherein
R¹ and R² are each independently selected chain moieties;
R³ and R⁴ are each independently hydrogen, absent, or a prodrug moiety;
L is a linking moiety, and pharmaceutically acceptable salts thereof.

75. The method of claim 74, wherein said apoptosis associated disorder is a neurodegenerative disorder.

76. The method of claim 74, wherein said apoptosis associated disorder is an eye disorder.

77. The method of claim 74, wherein said subject is a human.

78. A pharmaceutical composition comprising an effective amount of a phosphatidic acid compound and a pharmaceutically acceptable carrier, wherein said phosphatidic acid compound is of the formula (I): 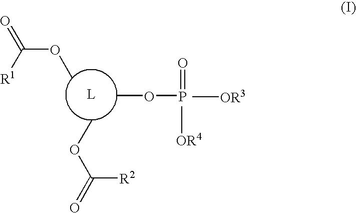
wherein
R¹ and R² are each independently selected chain moieties;
R³ and R⁴ are each independently hydrogen, absent, or a prodrug moiety;
L is a linking moiety, and pharmaceutically acceptable salts thereof.

79. The pharmaceutical composition of claim 78, wherein said effective amount is effective to treat a neurodegenerative disorder.

80. The pharmaceutical composition of claim 78, wherein said effective amount is effective to treat an eye disorder.
